SEQUENCE LISTING (2 sequences)

SEQ ID NO: 1 - RNA, 19 nt, artificial
  sample sequence (RNA)
ggcgauauucugcuacagu

SEQ ID NO: 2 - RNA, 19 nt, artificial
  sample sequence (RNA)
acuguagcagaauaucgcc

(19) United States
(12) Patent Application Publication
(10) Pub. No.: US 2010/0167410 A1
(43) Pub. Date: Jul. 1, 2010

(54) MASS-SPECTROMETRIC METHOD CARRIED OUT ON SAMPLES CONTAINING NUCLEIC ACIDS

(75) Inventors: Huseyin Aygün, Frankfurt am Main (DE); Michael Karas, Hattersheim (DE); Ute Bahr, Hattersheim (DE)

Correspondence Address: SMITH PATENT CONSULTING, LLC, 515 East Braddock Road, Suite B, ALEXANDRIA, VA, 22314, US

(73) Assignees: BioSpring Gesellschaft für Biotechnologie mbH, Frankfurt am Main (DE); Johann Wolfgang Goethe-Universität Frankfurt am Main, Frankfurt am Main (DE)

(21) Appl. No.: 12/518,152

(22) PCT Filed: Dec. 7, 2007
(86) PCT No.: PCT/EP2007/010663

§ 371 Date: Jun. 8, 2009

(30) Foreign Application Priority Data
Dec. 8, 2006 (EP) 06025448.9

(51) Int. Cl. G01N 33/00; G01N 1/28

(52) U.S. Cl. 436/94; 436/175

(57) ABSTRACT

The invention relates to a mass spectrometric method for the detection and for the quantification of double stranded nucleic acids which are not covalently associated with one another.

Nucleic acids are macromolecules composed of individual building blocks, the nucleotides. Alternating monosaccharides and phosphoric acid residues here form the basic structure of the nucleic acids, a nucleobase being linked to every monosaccharide. Important representatives of the nucleic acids are deoxyribonucleic acid (DNA), ribonucleic acid (RNA) and also derivatives and modifications thereof, such as the lock nucleic acid (LNA) which is a modified RNA. In addition, DNA-like and RNA-like organic polymers, such as peptide nucleic acid (PNA), are also often comprised by the generic term ‘nucleic acid’.

An essential property of the nucleic acids is that they cannot only be available as single strands of the polymer from nucleotides or derivatives thereof but also in the form of double strands or more complex forms (e.g. as a triple helix). Here, a special feature is that double stranded or more complex structures are not formed from any single strands of nucleic acids but the formation proceeds subject to the base sequence. Thus, a certain amount of complementarity in the base sequence of two single nucleic acid strands is necessary to be able to form a double-stranded structure. When there is sufficient complementarity, structures, such as a double stranded structure, form spontaneously after the nucleic acid single strands are brought together under physiological conditions (salt concentration, pH and temperature). Double strands are stabilized by hydrophobic interactions within the base stacking (stacking interactions) and by intermolecular hydrogen bridges of the respectively complementary bases of the single strands.

The prior art recovers double stranded nucleic acids by either separately synthesizing the complementary single strands followed by combining them into double strands under physiological conditions, or successively synthesizing the double strand in what is called a “tandem” synthesis of the single strand. In this connection, a linker introduced during the synthesis is hydrolyzed after the single strand synthesis following chemical processing and the resulting single strands having complementary base sequences are subsequently hybridized under physiological conditions.

In particular double stranded RNA molecules are of major interest lately. The technology referred to as RNAi (RNA interference) which uses short double stranded RNA sequences (so-called siRNAs) utilizes a special property of higher cells to regulate the intracellular protein expression on an mRNA level by means of such double strands and further cellular components. Elbashir and Tuschl were the first to show in 2001 that such a process can also be induced by means of short double stranded RNA molecules (siRNAs) (Elbashir S M, Lendeckel W, and Tuschl T, Genes Dev, 2001, 15(2), 188-200; Elbashir S M, Harborth J, Lendeckel W, Yalcin A, Weber K, Tuschl T, Nature, 2001, 411, 494-498; Caplen N J, Parrish S, Imani F, Fire A, and Morgan R A, Proc. Natl. Acad. Sci. U.S.A., 2001, 98, 9746-9747). Chemically produced double strands are usually concerned here which have a length of 19-21 base pairs and a dinucleotide as 3′-overhang (usually dTdT).

As to the successful application of double stranded structures in research and medicine it is particularly important to guarantee not only the purity of the single strands but also the completeness of the hybridization as a double strand. When used, an excess of single strands can result in numerous side-effects and should therefore be avoided, if possible. What is important in this connection is the ability of cells to recognize such single stranded nucleic acids via what is called TLR receptors on membrane surfaces and respond thereto by means of an immune response (e.g. by release of interferons). However, this immune response is undesired in particular for medical methods and should be avoided. As to RNA single strands it is above all the receptors TLR 7 and TLR 8 that are able to trigger such an immune response (Heil F, Hemmi H, Hochrein H, Ampenberger F, Kirschning C, Akira S, Lipford G, Wagner H, Bauer S, Science, 2004, 303, 1526-1529). Therefore, applications where double stranded nucleic acids act as an active substance should be well characterized with respect to the presence of excess single strands.

Equal amounts of both complementary single strands are usually required for the production of a double strand. In order to be able to achieve this quantitatively, nucleic acids, e.g. synthetic oligonucleotides, are quantified in the prior art by means of UV-VIS spectroscopy. For this, the coefficients of extinction of a solution which contains the nucleic acid are measured and the nucleotide content is calculated by means of the Lambert Beer Law. The coefficient of extinction of a nucleic acid is approximately composed additively of the coefficients of extinction of the single bases of a strand. However, an error of this additive assessment of the coefficient of extinction of the nucleic acid from the single bases is formed by neighborly interactions of the single bases, which changes the effective absorption of the single bases so that the coefficient of extinction of the total strand can no longer be calculated accurately. In addition, the nucleic acids can form intramolecular structures even under the measurement conditions, which further changes the coefficients of extinction of the single bases via what is called the hyperchromic effect so as to further falsify the calculated value of the oligonucleotide content. As a result, errors occur when the single strand concentration is determined. On account of the errors occurring when the single strand concentration of a sample is determined, the production of a double strand thus yields a product which along with the desired double strands contains an excess of a single strand. However, such samples are unfavorable since as pointed out above they trigger undesired side-effects, in particular in medical methods.

The resulting samples which along with the double strand amount may have another amount of single strand, must be characterized in particular by two measured values. On the one hand, these are the qualitative detection of the single strand excess (single strand A or complementary single strand B) and, on the other hand, the ratio of single strand to double strand in the sample.

The single strand amount is determined in the prior art via ion exchange or reverse phase HPLC. However, this process is complex, time-consuming and in addition calls for a constant optimization of the running conditions. In order to be able to also use this process quantitatively, pure samples of every single strand are required as a calibration standard. The detection limit is here within the range of 5% for the single strand amount. This can be explained inter alia by the fact that the constituents (strand A, B or double strand) often have similar retention times in the chromatography. An assessment thus becomes much more “complicated” and inaccurate as both components “flow into one another”. In addition, no direct statements on the excess single strand can be made by means of HPLC (strand A or strand B). When both strands have no or only an excessively low retention difference in the HPLC method used, the single strand excess must be determined via another method.

Another method of determining the ratio of single strand to double strand is the polyacrylamide gel electrophoresis (PAGE). It makes use of the circumstance that in the electrical field the migration properties of double strands and single strands are markedly different due to the charge conditions. This method is also dominated by numerous limitations. The accurate quantification of the excess amount—calibration substances are also required for this—as well as the characterization of the single strand available in excess prove to be defective and less favorable, in particular when both strands have equal length, thus showing a similar running behavior in the gel.

Another known method for the investigation of biomolecules is mass spectrometry (MS). It represents inter alia a simple and very efficient measuring method for determining the molecular mass of biomolecules (W. D. Lehmann: Massenspektrometrie in the Biochemie [mass spectrometry in biochemistry] Spektrum Akad. Verlag, Heidelberg 1996) and is predominantly used for the analysis of proteins and also nucleic acids.

Mass spectrometry is often used as an analytical method to check the quality of synthetically produced nucleic acids after the production. For example, a method is known which is referred to as LC-MS. Here, the liquid chromatography is linked with mass spectrometry. The liquid chromatography serves the purpose of separation and the mass spectrometry serves for identifying and/or quantifying the substances. Further detectors are usually added, such as UV-ELS detectors or conductivity detectors. For example, qualitative and quantitative information on contaminations in the oligonucleotide sample to be investigated can be identified by this. However, a technology referred as MALDI-MS (matrix assisted laser desorption ionization mass spectrometry) method is most frequently used (M. Karas, F. Hillenkamp, Anal. Chem. 1988, 60, 2299; M. Karas, U. Bahr, F. Hillenkamp, Int. J. Mass Spectrom. Ion Proc. 1989, 92, 231; M. Karas, U. Bahr, A. Ingendoh, F. Hillenkamp, Angew. Chemie 1989, 101, 805; F. Hillenkamp, M. Karas, Methods Enzymol. 1990, 193, 280). This technology is particularly suited for the rapid and qualitative investigation of oligonucleotides (Bonk T, Humeny A, The Neuroscientist, 2001, 7 (1), 6-12; Distler A M, Allison J, Anal Chem., 2001, 73 (20), 5000-5003). However, the prior art only describes few successful applications of this method where it was possible to detect intact biomolecules which are not stabilized via covalent interactions, such as nucleic acid double strands.

Causes regarding the difficulties of identifying double strands which are not covalently associated with one another by means of MALDI-MS, are the sample matrix used in the prior art which has an acidic pH of about 3 as well as the use of organic solvents. Both leads to the destabilization of nucleic acid double strands. Up to now, it is not known to what extent the laser intensity required for desorption (U.V. laser) contributes to destabilization.

Numerous variations have been developed to enable the detection of single strands which are not covalently associated in the MALDI mass spectrometry.

Lecci and Pannell (J. Am. Soc. Mass Spectrom., 1995, 6, 972-975) describe the use of 5-aza-2-thiothymine instead of 3-hydroxypicolinic acid as sample matrix to identify double stranded DNA by means of MALDI-MS. However, the modification of the sample matrix could not prevent that significant amounts of single strands—caused by the dissociation of the double strand—were measured during desorption. Thus, a statement on the ratio of excess single strand to the double strand of the sample cannot be made.

Little et al. (Int. J. Mass Spectrom. Ion Processes, 169/170, 1997, 323-330) describe the investigation of DNA by means of MALDI-MS where the sample matrix was additionally cooled to prevent the dissociation of the double strands. However, the cooling represents another complex process step which is unfavorable and could not prevent either that significant amounts of single strands were detected in the measurement. Thus, this process, too, does not provide the possibility of determining the ratio of excess single strand to the double strand of the sample.

Another method of investigating double-stranded nucleic acid samples by means of mass spectrometry was described by Kirpekar et al. (Kirpekar F, Berkenkamp S, Hillenkamp F, Anal. Chem., 1999, 71 (13), 2334-2339). Along with U.V. lasers and 6-aza-2-thiothymine as a sample matrix, an IR laser instead of a commercially available nitrogen laser was used here for the sample desorption. The sample material consisted of very long oligonucleotide double strands (70-mer and 80-mer), double strands of which were detected but not quantified. Thus, this method cannot furnish information on the ratio of excess single strand to double strand either.

Thus, there is a demand for a rapid, uncomplicated method of determining property parameters of a sample containing at least one biomolecule, preferably at least one nucleic acid, so as to be able to determine the property parameters, in the case of nucleic acid containing samples in particular the ratio of single strand to double strand of the nucleic acid of the sample and/or the excess of a single strand of nucleic acid in the sample.

It has surprisingly been found that certain property parameters of a sample which contains at least one biomolecule, preferably at least one nucleic acid, can be quantified by carrying out the below described method.

The invention relates to a method for determining at least one property parameter of a sample which contains at least one biomolecule, the method comprising the steps of:
(a) adding a standard to the sample;
(b) establishing a mass spectrum of the sample containing the standard under native conditions;
(c) establishing a mass spectrum of the sample containing the standard under denaturing conditions;
(d) comparing the peak height or peak area of at least one peak to be attributed to the biomolecule in the mass spectrum from step (b) with the peak height or peak area of the corresponding peak in the mass spectrum from step (c).

Based on the present invention, a biomolecule is understood to mean a chemical compound which in nature occurs in living organisms or is produced or can be produced by them. Biomolecules predominantly consist of carbon and hydrogen together with nitrogen, oxygen, phosphorus, sulfur and further, relatively rare elements. Where appropriate, the biomolecules consist, based on the present invention, of two non-covalently bound units, such as homodimeric or heterodimeric protein complexes or double stranded DNA, the latter being preferred.

In a preferred embodiment of the method according to the invention, the biomolecule comprises at least one nucleic acid, in particular one or more oligomolecules, e.g. a mixture of single stranded and/or double stranded nucleic acids.

Based on the present invention, “double strand amount” in the sample is understood to mean the ratio of the amounts (quantities of material) of the part of a nucleic acid which is available as a double strand to the part of a nucleic acid which is available as a single strand. The parts that are available as a double strand or a single strand, are in each case two complementary single strands which together may form a double strand. Thus, a double strand amount or a ratio of the particular single strand to the part available in the double strand in the sample can be determined for each of the complementary single strands which are preferably not identical.

DNA, RNA, non-natural derivatives thereof, such as LNA, and preferably naturally or synthetically produced nucleic acids, what is called oligonucleotides, can be used as nucleic acids contained in the sample.

In particular the double strand amount of the sample, the ratio of single strands to double strands in the sample, the excess of single strand in the sample, the interactive forces between different nucleic acids of the sample or the interaction of the nucleic acid available in the sample with other sample ingredients which do not contain nucleic acid can be determined as property parameters by the method according to the invention.

The method according to the invention is exemplified below for the measurement of nucleic acids, in particular double stranded and single stranded nucleic acids. However, it can correspondingly be applied to further biomolecules, such as proteins which are correspondingly available in complexes consisting of two non-covalently bound units, which would correspond to the double strand, and in corresponding monomers, which would correspond to the single strand.

“Comparison” of the peak heights or peak areas of individual peaks is understood to mean the relation or the conversion according to algebraic regulations, as described on the basis of this application, for example, of the numerical values which can be determined for the corresponding peaks from the spectra, e.g. count, area, etc. The quantification of peaks from spectra is known in the art.

Based on the present application, peaks that occur in several measurements and originate from the same substance are referred to as “corresponding”. Here, e.g. corresponding peaks are those which originate from a certain single strand that can only be seen under native conditions in the spectrum in the amount in which it is not bound in the double strand and under denaturing conditions yields a stronger signal since virtually no double strand is available any more. Based on the present invention, the corresponding peaks are preferably the peak pairs, to be attributed to a certain single strand each, of two measurements one of which is recorded under native conditions and the other under denaturing conditions.

Based on the present invention, “normalization” or “normalizing” is understood to mean the multiplication of the peak heights/areas of a spectrum with respect to another one with a factor determined such that a substance, preferably the standard, which was kept constant in both measurements, subsequently has the same peak height or area in both spectra. It is preferred for the method according to the invention to normalize all measurements with respect to a measurement, in particular with respect to the standard kept constant in the samples of all measurements.

The mass spectrum of a sample is obtained by the measurement of the sample in a mass spectrometer. A MALDI-based mass spectrometer is preferred as the mass spectrometer usable in the method according to the invention. In the MALDI mass spectrometer, the sample is introduced into the device in the form of what is called a matrix. The MALDI mass spectrometry is based above all on the co-crystallization of matrix and analyte with a molecular matrix excess of 100 to 100,000 times. Small organic molecules which strongly absorb at the employed laser wavelength (e.g. nitrogen laser at a wavelength of 337 nm) are usually selected as matrix substances. What follows is the excitation which after relaxation in the crystal lattice results in explosion-like particle separations on the surface of the matrix crystal by means of short high energy laser pulses having a pulse duration of some nanoseconds. A fragmentation of high-mass molecules is prevented by the combination with the matrix. The ionization mechanism in the MALDI mass spectrometry is not yet fully understood.

It has surprisingly been found that with particularly high concentrations of matrix buffer (diammonium hydrogen citrate, DAHC) and with optimum sample concentration as well as on avoidance of organic solvents, the double strand amount of a nucleic acid containing sample—even in a commercially available sample matrix, such as 6-aza-2-thiotymine (ATT)—is stably maintained during the desorption by means of U.V. laser. Thus, a quantification of the double strand amount in the sample is rendered possible.

The present invention is based inter alia on the problem that for the quantification of the content of double strand of a sample, e.g. consisting of two oligonucleotides, the signal intensities of the double strands and the single strands in the MALDI mass spectrum cannot directly be compared with one another since the resulting intensities depend on both the mass of the detected nucleic acid and the base composition per se.

A problem of the quantitative determination of the ratio of single strand to double strand and the determination of the excess amount of a single strand with respect to the complementary single strand is the variation of the absolute values of the peak heights/peak areas of a mass spectrometric measurement with respect to the next. These problems were solved by the introduction of a reference substance which is contained in the sample as an internal standard. Thus, a quantification of the ratio of single strand to double strand and the excess amount of a single strand was possible for the first time by means of the MALDI-MS technology.

In the method according to the invention, at least two measurements are carried out, one under native conditions where the double strand is stabilized and the other under denaturing conditions destabilizing the double strand. An intensity comparison of the peaks of the corresponding single strands in consideration of the internal standard here furnishes reliable information on the single strand amount of the sample. In particular, an internal standard is added in each case in two measurements, under native conditions and under denaturing conditions, of the dissolved sample and the single strand intensity (I_{SS}) is determined in relation to the intensity of the internal standard (I_{SS}). The single strand and double strand amounts can be calculated from the single strand intensities. Furthermore, it can be determined simultaneously what single strand is concerned in the excess if the masses of both strands differ (M_{strand A} ≠M_{strand B}).

For the measurement under “native” conditions, i.e. under optimum MALDI mass spectrometry conditions where in particular also the double strands are maintained and can be quantified as such, an aqueous solution is initially produced which consists of the oligonucleotide sample as well as an added standard. The standard which comprises another oligonucleotide, for example, may not interact with the sample under the experimental conditions, in particular it may not interact under “native” and “denaturing” conditions. The added standard here functions as an internal standard.

Another measurement is then carried out under “denaturing” conditions. For this, a denaturing agent, e.g. formic acid, is added to the aqueous solution of the sample which consists of the nucleic acid along with the added standard. The denaturing agent is weighted and added in amounts (preferably 10-30% by weight, in particular 15-25% by weight, e.g. 20% by weight of the sample) such that the double strand fully decomposes into single strands. The measurement previously carried out under “native” conditions is thus repeated under identical conditions only with the addition of denaturing agent.

Since the concentration of the standard is identical in both measurements, a comparison of one measurement under “native” conditions and the other measurement under “denaturing” conditions is enabled in consideration of the ratio of the intensities of the signals for the standard. For this, a correction factor y is introduced which reflects the ratio of the intensities of the standard of both measurements.

$$y = \frac{{I}_{standard 1}}{{I}_{standard 2}} \qquad ( 1 )$$

The measurement under “native” conditions discloses the intensity of the single strand which is not bound in the double strand (I_{SSnative}). The measurement under “denaturing” conditions discloses the intensity and thus the amount of the single strand contained in the sample on the whole (I_{SSdenat}). Considering the ratio factor y between both different measurements, this serves for determining the amount of the single strand at which it is present under “native” conditions (I_{SSnative} (%)). Since this amount is supplemented with the amount of single strand which is available in bound form in the double strand to give 100%, the amount of single strand which is available in bound form in the double strand can be determined therefrom (I_{DS} (%)).

$$\begin{matrix}{I}_{SSnat} ( \% ) = y \cdot \frac{{I}_{SSnat}}{{I}_{SSdenat}} \cdot 100 & ( 2 ) \\ {I}_{DS} ( \% ) = 100 - {I}_{SSnat} ( \% ) & ( 3 )\end{matrix}$$

For the purpose of quantification, the peak heights can be used along with the peak areas provided that standard and single strands have a similar mass range so that the resolution (peak width) is equal for both.

All single stranded oligonucleotides which show no interaction with the analyte substances under the experimental conditions and do not change under the influence of the denaturing agent are suitable as a standard substance. The mass range of the standard can also readily be matched with the single strands by the use of oligonucleotides, which as pointed out above results in a comparable dissolution. A difference of 1-2 nucleotide building blocks between the length of the oligonucleotides and the length of the single strands to be investigated is favorable.

Such an analysis of the resulting spectra presupposes a linearity of the mass spectrometric measurement over two concentration orders of magnitude, i.e. the concentrations of the single strands are between 1 and 100 percent of the measurement range of the mass spectrometer. Furthermore, attention has to be paid to the fact that in the measurement under “denaturing” conditions—as compared to the measurement under “native” conditions—the ratio of ion intensity of standard and analyte may not be influenced to avoid a falsification of the results of both measurements.

Along with the above indicated determination of the amount in which a single strand is bound in the double strand, a possibly existing excess of one of both single strands (strand 1 or 2) which form the double strand can be determined from both measurements under “native” and “denaturing” conditions. The direct difference between the signal intensities of both strands within a spectrum cannot be directly considered for this since on account of different ionization and detection probabilities of both molecules the excess cannot be obtained directly, e.g. by subtraction of the intensities. The different ionization and detection probability of a molecule is considered by means of an unknown response factor z. Since the response factor z of a strand remains constant with respect to the other between two measurements, e.g. under “native” and “denaturing” conditions, this unknown factor z can be eliminated via the ratio of the intensities between the measurements. In this way, the excess of a single strand can be quantified with respect to the other.

$${I}_{1 D} = z ( {I}_{2 D} + {I}_{Y} )$$

$${I}_{1 N} = z ( {I}_{2 N} + {I}_{Y} )$$

$$\frac{{I}_{1 D}}{{I}_{2 D} + {I}_{Y}} = \frac{{I}_{1 N}}{{I}_{2 N} + {I}_{Y}}$$

$${I}_{Y} = \frac{{I}_{1 D} \cdot {I}_{2 N} - {1}_{1 N} \cdot {I}_{2 D}}{{I}_{1 N} - {I}_{1 D}}$$

wherein I_{y} represents the excess of a single strand, z is the unknown response factor, I_{1D} and I_{2D} are the standardized intensities of the single strands 1 and 2 under “denaturing” conditions and I_{1N} and I_{2N} represent the intensities of the single strands 1 and 2 under “native” conditions.

In the case of an excess of single strand 1, I_{Y} is positive; in the case of an excess of single strand 2, I_{Y} is negative and when there is no excess of a single strand I_{Y}=0.

Finally, with respect to an excess of single strand 1 the relative excess of single strand 1 (I_{Y1} (%)) can be calculated according to equation (4a) and as regards an excess of single strand 2 the relative excess of single strand 2 (I_{Y2} (%)) can be calculated according to equation (4b).

$$\begin{matrix}{I}_{Y 1} ( \% ) = \frac{{I}_{Y}}{{I}_{Y} {I}_{Y}} \cdot 100 & ( 4 a ) \\ {I}_{Y 2} ( \% ) = \frac{\vert {I}_{Y} \vert}{{I}_{2 D} - \vert {I}_{Y} \vert} \cdot 100 & ( 4 b )\end{matrix}$$

The presented method according to the invention of the double strand quantification has in particular the advantage that the peak ratios are determined in a single solution. Fluctuations of the measurement parameters are eliminated by the relation of the intensities of the peaks of two measurements so as to ensure a high accuracy of the values to be determined.

Furthermore, the method according to the invention has the advantages that on account of the use of a MALDI mass spectrometer the measurement is very fast since the method can be automated and the sample throughput is very high. Thus, it typically only takes some minutes to record both measurements under “native” and “denaturing” conditions. The evaluation of the spectra and the corresponding calculations of the intensities as well as the excess ratio can easily be made in a computer-assisted way. Except for an internal standard which may always be the same, the use of calibration substances is not necessary. Thus, the error in the determination of the double strand amount can be lowered to below 2 percent on account of the above advantages.

The method according to the invention can be carried out with every MALDI mass spectrometer as long as it is a linear MALDI mass spectrometer.

The MALDI sample matrixes usable in the method according to the invention are neutral matrixes which do not result in a denaturation of the double strands. Saturated solutions of 6-aza-2-thiothymine (ATT, available from Fluka, Germany) (about 6-7 mg/ml) in 100-200 mM, more advantageously 120-150 mM, aqueous diammonium hydrogen citrate solution (DAHC, available from Fluka, Germany) have proved to be particularly favorable. The concentration of the detectable double strand increases with a DAHC concentration increasing from 0 mM and approaches saturation at 150-200 mM (cf. example 2). Precipitation of dissolved substances may occur from 200 mM DAHC. 2 μL of this matrix solution are typically mixed with 0.5 μL of an oligonucleotide solution of about 20 μM directly on the sample plate and dried in a cold air current. According to the invention, the non-use of organic solvents, such as acetonitrile, is particularly important for the MALDI sample preparation, which are usually used in the prior art to improve the solubility of the samples. The effective sample concentration of about 20 μM is within the range of the usually used ones, a reduction of the sample concentration effecting a decreasing stability of the double strand. In order to achieve a sufficient signal-to-noise ratio, 50-100 individual spectra are typically accumulated.

The method according to the invention is particularly favorable at a nucleic acid length from 10 to 60 bp, in particular 18-23 bp, e.g. of siRNA. A lower limit for a sufficient stability of the double strand might be about 10 bp.

Furthermore, the invention relates to the use of an acid for the denaturation of nucleic acids in the sample preparation for mass spectrometry.

The invention also relates to the use of a mixture of 6-aza-2-thiothymine (ATT) and diammonium hydrogen citrate (DAHC) in a sample matrix for the MALDI mass spectrometry.

The method according to the invention is also suitable for the detection of other, non-covalent interactions between nucleic acid single strands which have complementary base sequences. Thus, a conclusion on the binding energy for forming the double strand from the particular strands can be drawn via the ratio between species available as a single strand and species available as a double strand. Corresponding methods for the determination of the binding energies from the binding ratios are known in the art.

In addition, the method according to the invention can be used to determine protein interactions since in accordance with the above statements the ratio between dissociated proteins and protein complexes can be determined in an identical way. Conclusions can be drawn from the ratio determined by means of the method according to the invention to corresponding interaction energies.

In addition, the method according to the invention can be used for the quality control of synthetically produced, double stranded oligonucleotides as occur in gene expression studies, for example (siRNA or RNAi).

Finally, the method according to the invention can favorably be used for the production of mixtures having a defined ratio between double strand and single strand. This is possible by means of mixture titration with subsequent MALDI mass spectrometry according to the invention, for example. Since the single strand excess can rapidly and readily be determined for every titration stage, a simple method is thus provided to obtain compounds which comprise a defined amount of double strand, preferably mainly of double strand, e.g. >98%. Such compounds are of major significance in particular for pharmaceutical applications.

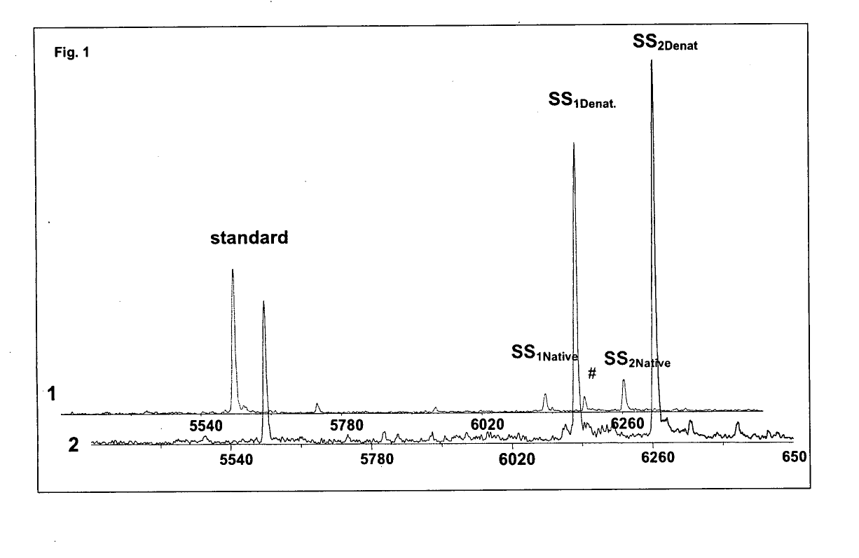

FIG. 1 shows a diagram having two offset mass spectra of a sample under native and denaturing conditions.

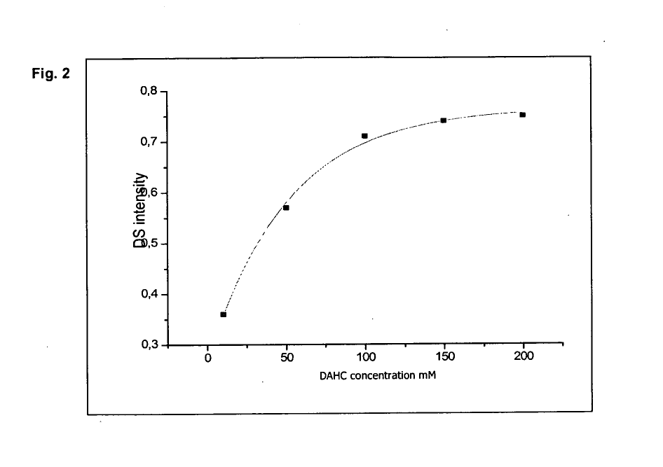

FIG. 2 shows a diagram where the double strand/single strand intensity of a nucleic acid containing sample is plotted against the DAHC concentration of the sample solution.

The invention is further illustrated by means o the following examples:

### Example 1

### Quantification of the Double Strand Amount

FIG. 1 shows two offset MALDI-MS spectra, lane 1 having been recorded under “native” conditions and lane 2 under “denaturing” conditions. Only the mass range of the standard and the single strands is shown since the peaks of the double strand are not required for the assessment. An oligonucleotide strand was used as a standard which does not interact with the complementary single strands 1 and 2 (SS₁ and SS₂) to be investigated. Since the standard in the samples of both measurements is identical, the intensities of both spectra can be normalized via the correction factor y which is determined according to the above formula (1) so that then the intensity of the standard is equal in both spectra and the respective single strand intensities thus become directly comparable. An intensity of 756 counts under native conditions and 13227 counts under denaturing conditions was measured for the spectrum shown in FIG. 1 after the normalization on the standard for single strand 1 (SS₁). According to the above formula (3) there is a double strand amount, based on the single strand 1, of 100 −756/13227×100=94.3%.

Furthermore, a possible excess of a single strand can be determined. Along with the known values for single strand 1 (SS₁), the values of the single strand 2 (SS₂) of the peaks are also required under “native” conditions (1347 counts) and “denaturing” conditions (16749 counts). When inserted in the above mentioned formula (4b), a relative excess for single strand 2 of 2.5% thus results. This means that a total of 5.7% of single strand 1 is available in the form of the single strand and 8.2% of single strand 2 is available in the form of the single strand, single strand 2 having a relative excess of 2.5%.

A saturated solution of 6-aza-2-thiothymine (ATT) in 100-200 mM aqueous diammonium hydrogen citrate solution (DAHC) was used in the investigations as a sample matrix. 2 μL of this matrix solution were directly mixed with 0.5 μL oligonucleotide solution (about 20 μM) on the sample plate and dried in a cold air current.

Sample sequence (RNA):
5′ G*GC*GA*UA*UU*CU*GC*UA*CA*GU*
x ACUGUAGCAGAAUAUCGCC 3′
(* = 2′ Omethyl)

An RNA oligonucleotide having a length of 17 base pairs was used as a standard which did not interact with the sample under the experimental conditions. The mass spectrometric analysis was carried out with a MALDI time of flight mass spectrometer (Voyager De-Pro, Applied Biosystems, Framingham, U.S.A.) in the linear operation. Spectra of the positive ions and negative ions can be used for the analysis since both show comparable intensities and dissolutions. An acceleration voltage of 20 kV, a grid voltage of 95% of the acceleration voltage and a delay time of 600 ns were selected as operating parameters. In order to obtain a sufficient signal-to-noise ratio, 50-100 individual spectra were accumulated for each spectrum. In order to calculate the peak areas and heights, the spectra were 19 pt smoothed and the base line was corrected.

### Example 2

### Single Strand/Double Strand Ratio at Different DAHC Concentrations

In FIG. 2, the ratio of single strand to double strand was investigated for various

DAHC concentrations. The given values for the double strand intensity are arbitrary units and do not correspond to the real ratios.

It has been found that the concentration of double strand increases with increasing DAHC concentration; it approaches saturation at 150-200 mM.

The samples were prepared according to Example 1 and measured. The ratios of double strand to single strand were calculated according to the above mentioned process of the spectra measured according to Example 1.

1. A method for determining at least one property parameter of a sample which contains at least one nucleic acid, the property parameter being selected from the group consisting of double strand amount of the sample, ratio of single strands to double strands in the sample, and excess of a single strand with respect to a second single strand in the sample, the method comprising the steps of:
(a) adding a standard to a sample containing at least one nucleic acid;
(b) establishing a mass spectrum of the sample containing the standard under native conditions, said mass spectrum including at least one peak attributable to said at least one nucleic acid;
(c) establishing a mass spectrum of the sample containing the standard under denaturing conditions, said mass spectrum including at least one peak attributable to said at least one nucleic acid;
(d) comparing the peak height or peak area of the at least one peak attributable to the nucleic acid in the mass spectrum from step (b) with the peak height or peak area of the corresponding peak in the mass spectrum from step (c) to determine a property parameter of said sample.

2. The method according to claim 1, wherein said mass spectrum from steps (b) and (c) further comprise at least one peak attributable to said standard, further wherein the ratio of the peak signals corresponding to the standard in the mass spectra from steps (b) and/or (c) is considered with respect to one another.

3. The method according to claim 1, characterized in that the property parameter is the double strand amount of the sample.

4. The method according to claim 1, characterized in that the property parameter is the ratio of single strands to double strands in the sample.

5. The method according to claim 1, characterized in that the property parameter is the excess of a single strand with respect to a second single strand in the sample.

6. The method according to claim 1, characterized in that the denaturing conditions are produced by the addition of a denaturing agent, preferably by the addition of an acid.

7. The method according to claim 1, characterized in that the nucleic acid is DNA.

8. The method according to claim 1, characterized in that the nucleic acid is RNA.

9. The method according to claim 1, characterized in that the mass spectrometer is based on MALDI.

10. The method according to claim 9, characterized in that the MALDI sample matrix comprises a double strand stabilizing composition.

11. The method according to claim 9, characterized in that the MALDI sample matrix comprises 6-aza-2-thiothymine (ATT) and diammonium hydrogen citrate (DAHC).

12. The method according claim 1, characterized in that the standard is a single stranded nucleic acid.

13. A method of preparing a sample which contains at least one nucleic acid for mass spectrometry, said method comprising the step of adding formic acid to said sample so as denaturize the at least one nucleic acid in the sample, wherein said formic acid is added in an amount such that it is available in a concentration of 10-30% by weight of the sample.

14. A method of preparing a sample matrix for MALDI mass spectrometry, said method comprising the step of adding a mixture of 6-aza-2-thiothymine (ATT) and diammonium hydrogen citrate (DAHC) to the sample matrix for the MALDI mass spectrometry, wherein the DAHC used is in a concentration of 50-250 mM.

15. The method of preparing a sample matrix for MALDI mass spectrometry according to claim 14, characterized in that DAHC used is in a concentration of 100-200 mM.

16. The method of preparing a sample matrix for MALDI mass spectrometry according to claim 14, characterized in that said sample matrix comprises an optionally saturated solution of ATT is prepared in aqueous DAHC solution.

17. The method of preparing a sample matrix for MALDI mass spectrometry according to claim 16, characterized in that a sample is mixed with the sample matrix and the mixture is dried in an air current before mass spectrometry is carried out.

18. The method of preparing a sample matrix for MALDI mass spectrometry according to claim 14, characterized in that no organic solvents are used in the MALDI sample preparation.
